(19) United States
(12) Patent Application Publication
(10) Pub. No.: US 2026/0224820 A1
(43) Pub. Date: Aug. 6, 2026

(54) MEDICAL DEVICE HANDLE WITH ERGONOMIC GRIP

(71) Applicants: Boston Scientific Scimed, Inc., Maple Grove, MN (US); Boston Scientific Medical Device Limited, Galway (IE)

(72) Inventors: Yin XIA, Shanghai (CN); Kensuke HAYASHI, Penang (MY); Maria Therese MAILLET, Hudson, MA (US)

(73) Assignees: Boston Scientific Scimed, Inc., Maple Grove, MN (US); Boston Scientific Medical Device Limited, Galway (IE)

(21) Appl. No.: 19/464,904

(22) Filed: Jan. 30, 2026

(30) Foreign Application Priority Data
Feb. 1, 2025 (CN) 202510127688.8

(51) Int. Cl. A61M 5/34

(52) U.S. Cl. CPC A61M 5/34; A61M 2205/586

(57) ABSTRACT

A medical device handle comprises a first body defining a lumen a second body having a portion that is movably received within the lumen. The second body may include a grip. The grip may include a proximal rib, a distal rib, a middle rib, a first plurality of intermediate ribs between the proximal rib and the middle rib, and a second plurality of intermediate ribs between the middle rib and the distal rib. Each of the first plurality of intermediate ribs and the second plurality of intermediate ribs may have an arcuate profile that defines a concave outer shape of the grip. The second body may be configured to move proximally and distally along a central longitudinal axis of the second body in order to actuate a needle of a medical device.

### CROSS-REFERENCE TO RELATED APPLICATIONS

This application claims the benefit of priority under 35 U.S.C. § 119 from Chinese Application No. 202510127688.8, filed Feb. 1, 2025, which is incorporated by reference herein in its entirety.

### TECHNICAL FIELD

This disclosure relates generally to medical devices, and more particularly to ergonomic handles for medical devices.

### BACKGROUND

Injection needles may include a handle for gripping by an operator and a shaft for insertion into a body of a subject (e.g., via an insertion device such as a scope). A lumen of the shaft may receive a needle that may be selectively advanced or retracted from a distal tip of the shaft. The needle may define a lumen through which a fluid, such as contrasting agent, saline, or another agent (e.g., a lifting agent) may be injected. The needle may have a distal opening that may be used to deliver the fluid to a treatment site within a body of the subject.

The handle may include components for controlling the needle (e.g., advancing and retracting the needle), as well as components for coupling to a source of fluid. A need exists for ergonomic handles for medical devices, such as injection needles.

### SUMMARY

Disclosed are handles for medical devices, such as injection needles. The handles have ergonomic features to, among other things, accommodate a range of user hand sizes, increase friction with a user's hand, and inhibit the handle from rolling on a tabletop.

In an example, a medical device handle comprises a first body defining a lumen a second body having a portion that is movably received within the lumen. The second body may include a grip. The grip may include a proximal rib, a distal rib, a middle rib, a first plurality of intermediate ribs between the proximal rib and the middle rib, and a second plurality of intermediate ribs between the middle rib and the distal rib. Each of the first plurality of intermediate ribs and the second plurality of intermediate ribs may have an arcuate profile that defines a concave outer shape of the grip. The second body may be configured to move proximally and distally along a central longitudinal axis of the second body in order to actuate a needle of a medical device.

Any of the aspects disclosed herein may have any of the following features, alone or in any combination. At least some of the first plurality of intermediate ribs may have a radially outer surface that is planar and has a non-zero angle with respect to the central longitudinal axis. Each of the first plurality of intermediate ribs and the second plurality of intermediate ribs may include a midpoint. At least one of the angled surfaces of the first plurality of intermediate ribs may be angled toward the midpoint of the first plurality of intermediate ribs. The radially outer surface a rib of the first plurality of intermediate ribs may have a proximalmost end that is a first distance from the central longitudinal axis and a distalmost end that is a second distance from the central longitudinal axis, wherein the first distance differs from the second distance. The second plurality of intermediate ribs collectively may have an arcuate profile that defines a concave outer shape of the grip. The proximal rib, the distal rib, and the middle rib may extend further from the central longitudinal axis than any of the intermediate ribs of the first plurality of intermediate ribs. The proximal rib may be a first proximal rib. The distal rib may be a first distal rib. The middle rib may be a first middle rib, wherein the first body includes a second proximal rib, a second distal rib, a second middle rib. A third plurality of intermediate ribs may be between the second proximal rib and the second middle rib, A fourth plurality of intermediate ribs may be between the second middle rib and the second distal rib. The third plurality of intermediate ribs collectively may have an arcuate profile that defines a concave outer shape of the first body. The fourth plurality of intermediate ribs collectively may have an arcuate profile that defines a concave outer shape of the first body. The third plurality of intermediate ribs of the first body may have a greater change in height between tallest and shortest ribs of the third plurality of intermediate ribs compared to a change in height between tallest and the shortest ribs of the first plurality of intermediate ribs of the second body. The second proximal rib and the second distal rib of the first body may have different heights. The first proximal rib and the first distal rib of the second body may have a same height. The second middle rib of the first body may have a same height as the second proximal rib of the first body. A height of a rib of the first plurality of intermediate ribs of the second body that is adjacent to the middle rib may be the same height as a rib of the second plurality of intermediate ribs of the second body adjacent to the middle rib. Each of the first plurality of intermediate ribs and the middle rib may include a planar distal face and a planar proximal face. The proximal rib, the distal rib, and the middle rib may extend further from the central longitudinal axis than any of the intermediate ribs of the second plurality of intermediate ribs.

In another aspect, A medical device handle may comprise a first body defining a lumen. The first body may include a plurality of ribs extending radially relative to a central longitudinal axis of the handle. The plurality of ribs may include a distal rib, a proximal rib, and a middle rib; a first plurality of intermediate ribs positioned between the distal rib and the middle rib; and a second plurality of intermediate ribs positioned between the middle rib and the proximal rib. Each of the first plurality of intermediate ribs and the second plurality of intermediate ribs may have an arcuate profile that defines a concave outer shape of the first body. The handle may comprise a second body having a portion that is movably received within the lumen. The second body may be configured to move proximally and distally along the central longitudinal axis in order to actuate a needle of a medical device.

Any of the disclosed aspects may have any of the following features, alone or in any combination. At least some of the second plurality of intermediate ribs may have a radially outer surface that is planar and has a non-zero angle with respect to the central longitudinal axis. The second plurality of intermediate ribs may have an arcuate profile that defines a concave curve. Each of the distal rib, the proximal rib, and the middle rib may extend further from the central longitudinal axis than any of the ribs of the first plurality of intermediate ribs and the second plurality of intermediate ribs. At least some of the first plurality of intermediate ribs and the second plurality of intermediate ribs may have a radially outer surface that is planar and has a non-zero angle with respect to the central longitudinal axis. Each of the first plurality of intermediate ribs and the second plurality of intermediate ribs may include a midpoint. At least one of the angled surfaces of the first plurality of intermediate ribs may be angled toward the midpoint of the first plurality of intermediate ribs.

In another aspect, a medical device handle may comprise a first body defining a lumen. The body may include a first plurality of ribs extending radially outward relative to a central longitudinal axis of the handle. The first plurality of ribs may form a first arcuate profile and a second arcuate profile. The second arcuate profile may be proximal to the first arcuate profile. The handle may further comprise second body having a portion that is movably received within the lumen. The second body may include a second plurality of ribs extending radially outward relative to the central longitudinal axis. The second plurality of ribs may form a third arcuate profile and a fourth arcuate profile. The fourth arcuate profile may be proximal to the third arcuate profile.

The foregoing general description of the illustrative embodiments and the following detailed description thereof are merely exemplary aspects of the teachings of this disclosure and are not restrictive.

### BRIEF DESCRIPTION OF THE DRAWINGS

The accompanying drawings, which are incorporated in and constitute a part of this specification, illustrate aspects this disclosure and together with the description, serve to explain the principles of the disclosure.

FIG. 1A depicts a perspective view of an exemplary medical device in a first configuration.

FIG. 1B depicts a perspective view of the medical device of FIG. 1A in a second configuration.

FIG. 2 depicts a cross-sectional view of a portion of a handle of the medical device in the first configuration.

FIG. 3 depicts a side view of a distal body of the handle of the medical device.

FIG. 4 depicts a side view of a proximal body of the handle of the medical device.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

It may be understood that both the foregoing general description and the following detailed description are exemplary and explanatory only and are not restrictive of the invention, as claimed. As used herein, the terms “comprises,” “comprising,” “has,” “having,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator, and the term “proximal” refers to a direction toward an operator. The term “exemplary” is used in the sense of “example,” rather than “ideal.” The term “approximately,” or like terms (e.g., “substantially” or “about”), includes values +/−10% of a stated value. Unless otherwise stated, ranges disclosed herein include the end points of the ranges.

Medical devices, such as injection needles, may include a handle for being gripped by an operator and a shaft distally extending from the handle. Although injection needles are referred to herein, aspects of the handles described herein may be utilized with other types of medical devices, such as endoscopic medical accessory devices. The shaft may be configured to be inserted into a body of a subject (e.g., via an introduction device such as an endoscope or other type of scope). A feature/element at the distal end of the shaft (e.g., a needle) may be operable to perform a procedure at a treatment site within the body of the subject. The handle may include a first body and a second body. The first body may define a lumen, in which a portion of the second body may be movably received. The second body may be extended and retracted relative to the first body in order to control the feature at the distal end of the shaft. The second body may include a barrel or grip for grasping by the operator in order to extend or retract the second body. The first body may include a barrel or grip for receiving fingers of the operator. The second body and the first body may be configured to be used by a variety of operators, and operators may hold the handle in various manners. The handle may be comfortable for operators with varying hand sizes to hold and actuate, and the handle may include one or more features that facilitate an operator in locating a grip position, for example, without seeing the handle. Furthermore, the first body and second body may include features to help prevent slippage within the operator's hand by, for example, increasing friction. A proximal end of the second body may include an interface for receiving and securing a syringe or other source of fluid.

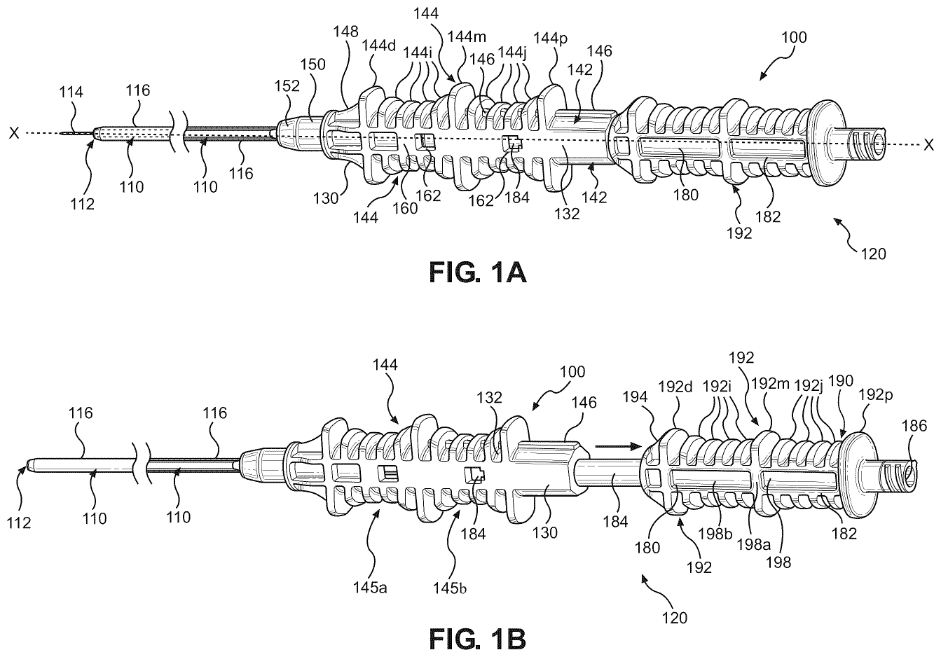

FIG. 1A shows a medical device 100 in a first configuration. FIG. 1B shows medical device 100 in a second configuration. With particular reference to FIG. 1A, medical device 100 may include a shaft 110 and a handle 120 (e.g., a medical device handle). Shaft 110 and handle 120 may extend along a central longitudinal axis of medical device 100, an axis X. Shaft 110 may have a distal end 112 with an element/feature configured to be operated by handle 120. For example, shaft 110 may have a needle 114 at distal end 112 of shaft 110. Needle 114 may be movable with respect to a sheath 116 of shaft 110, such that needle 114 may be extended from (e.g., to a position distally beyond) distal end 112 of sheath 116. Further, needle 114 may be retracted relative to sheath 116 (e.g., to a position proximal of a distal end of sheath 116) such that needle 114 is covered by sheath 116.

Needle 114 or another element (e.g., a hypotube) may extend through sheath 116 of shaft 110 to a proximal end of shaft 110. Needle 114 (or another element) may have a lumen (not shown) extending therethrough. As discussed below, a portion of handle 120 may be coupled to needle 114 or another element, and needle 114 or another element may be controlled with handle 120. Although a needle 114 is described herein, it will be appreciated that medical device 100 may include other types of features at its distal tip (e.g., basket, snare, forceps, stapler, etc.), and medical device 100 is not limited by a type of instrument at its distal tip.

Handle 120 may include a first body 130, which may be a distal portion of handle 120. First body 130 may be fixed with respect to sheath 116. A distal end of handle 120 or first body 130 may include a strain relief 150 radially surrounding shaft 110/sheath 116. A distal end of strain relief 150 may include a tapered portion 152 decreasing in diameter in a proximal to distal direction.

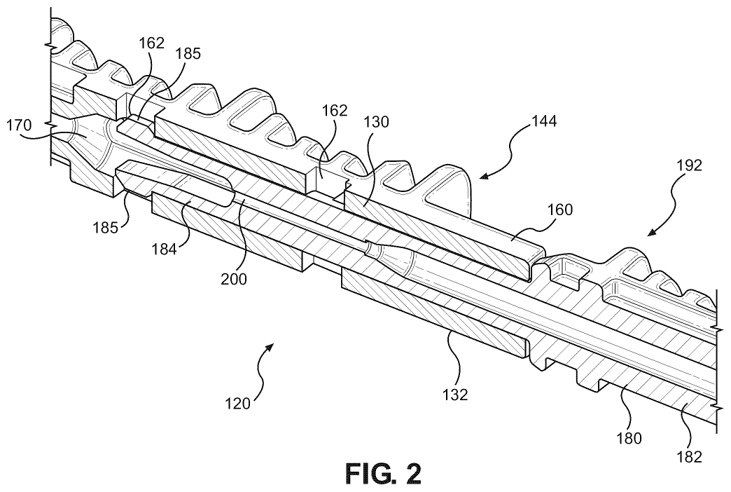

Handle 120 may further include a second body 180, which may be a proximal portion of handle 120. First body 130 may define a lumen 170 (shown in FIG. 2) for receiving a portion of second body 180 therein. For example, second body 180 may include a shank 184 (shown in FIGS. 1B and 2) that is movably received within lumen 170. Second body 180 may be akin to a plunger and may be movable proximally and distally relative to first body 130 and shaft 110/sheath 116. As second body 180 moves proximally and distally, needle 104 may be retracted and advanced, respectively, for example, relative to sheath 116. For example, shank 184 or another element of second body 180 may be directly or indirectly coupled to needle 114.

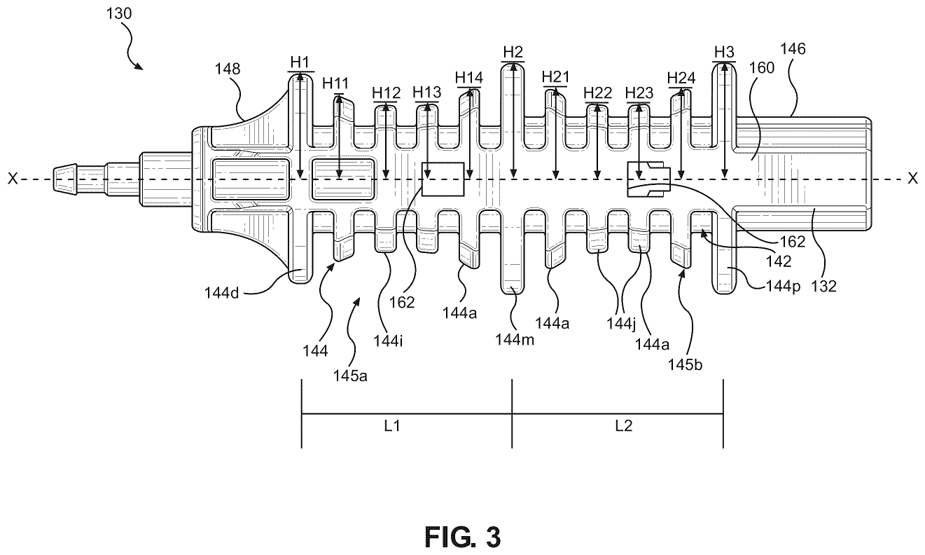

As shown in FIGS. 1A, 1B, and 3, in particular, first body 130 may include a barrel 132 (e.g., a grip). A proximal portion of barrel 132 may serve as a finger rest for one or more fingers of an operator's hand. Barrel 132 may have an overall cylindrical shape, with flattened sides, as discussed below. In some aspects, a central longitudinal axis of barrel 132 may be approximately coaxial with axis X of handle 120. Barrel 132 may have a diameter of approximately 10 mm, measured perpendicularly to axis X. Lumen 170 may extend longitudinally through barrel 132, as shown in FIG. 2.

Barrel 132 may form two approximately planar sides 160 of first body 130. Planar sides 160 may be smooth surfaces extending along the length of first body 130 parallel to axis X. On two ribbed sides 142 of first body 130, a plurality of ribs 144 may extend radially outward, relative to axis X, from barrel 132. These ribs 144 may provide enhanced grip and tactile feedback for the operator during use of medical device 100, among other benefits. In some implementations, ribbed sides 142 and planar sides 160 may alternate around the perimeter of first body 130 relative to axis X, creating a varied surface texture that may accommodate different gripping preferences or hand positions. The two ribbed sides 142 and the two planar sides 160 may be arranged about the circumference of first body 130 in an alternating pattern. Explained differently, the two planar sides 160 may be positioned opposite to one another about axis X, and the two ribbed sides 142 may be positioned opposite one another about axis X. When medical device 100 is positioned on a substantially flat or angled surface (e.g., a table), planar sides 160 and ribbed sides 142 may help to inhibit medical device 100 from rolling on the surface.

Ribbed sides 142 may include ribs 144 extending radially outward from a surface of barrel 132 relative to axis X. Ribs 144 on each of ribbed side 142 may include a distal rib 144d, a proximal rib 144p, a middle rib 144m, intermediate ribs 144i (a first plurality of intermediate ribs) positioned between distal rib 144d and middle rib 144m, and intermediate ribs 144j (a second plurality of intermediate ribs) between middle rib 144m and proximal rib 144p. Because each of two ribbed sides 142 includes plurality of ribs 144, first body 130 have two distal ribs 144d, two proximal ribs 144p, two middle ribs 144m, and two sets of intermediate ribs 144i (first and third pluralities of intermediate ribs), and two sets of intermediate ribs 144j (second and fourth pluralities of intermediate ribs). Ribs 144 of first ribbed side 142 may be identical or substantially identical to corresponding ribs 144 of second ribbed side 142. In some aspects, ribs 144 may have varying heights, with distal rib 144d, proximal rib 144p, and middle rib 144m extending further radially outward than intermediate ribs 144i and 144j. Each rib of ribs 144 may include a planar distal face and a planar proximal face. Together, ribs 144 may define a curved outer surface of ribbed sides 142, as described below. Together, distal rib 144d, middle rib 144m, and intermediate ribs 144i may define a first grip portion 145a. Middle rib 144m, proximal rib 144p, and intermediate ribs 144j may define a second grip portion 145b.

As shown in FIG. 3, distal rib 144d and middle rib 144m may define a distance or length, L1 (approximately parallel to axis X), extending from distal rib 144d to middle rib 144m. Length L1 may be a length of first grip portion 145a. Length L1 may be approximately 10 mm to approximately 30 mm, approximately 15 mm to approximately 25 mm, or approximately 20 mm. The values of L1 above may optimize a size of the grip defined by intermediate ribs 144i, distal rib 144d, and middle rib 144m. For example, these values of L1 may provide good grip to a high number of individuals with varying hand sizes.

Further, middle rib 144m and proximal rib 144p may define a distance or length, L2, extending from middle rib 144m to proximal rib 144p. Length L2 may be a length of second grip portion 145b. Length L2 may be approximately 10 mm to approximately 30 mm, approximately 15 mm to approximately 25 mm, or approximately 20 mm. The values of L2 above may optimize a size of the grip defined by intermediate ribs 144i, middle rib 144m, and proximal rib 144p. For example, these values of L1 may provide good grip to a high number of individuals with varying hand sizes. In some examples, lengths L1, L2 (and, thus, lengths of grip portion 145a and grip portion 145b) may be the same, to accommodate the similar sizes of the fingers that are typically positioned on grip portion 145a and grip portion 145b.

Grip portions 145a and 145b may each have an overall arcuate, concave shape (e.g., a concave outer shape). In other words, first plurality of intermediate ribs 144i may collectively have an arcuate profile that defines a concave outer shape of the grip (e.g., grip portion 145a) and second plurality of intermediate ribs 144j may collectively have an arcuate profile that defines another concave outer shape of the grip (e.g., grip portion 145b). Such a shape may result from the varying heights (e.g., an arcuate profile) of intermediate ribs 144i, 144j discussed in further detail below. The concave shapes of grip portions 145a and 145b may be chosen to provide a comfortable grip to a wide range of users. For example, users with smaller hands may prefer to hold grip portions 145a or 145b near centers (narrowest) portions of grip portions 145a and 145b, respectively. Additionally or alternatively, users may use the angled proximal or distal portions of grip portions 145a, 145b to provide additional support/a stronger hold on grip portions 145a, 145b. In other words, intermediate ribs 144i nearer to the midpoint of grip portion 145a may have a lesser height than intermediate ribs 144i positioned further from the midpoint of grip portion 145a. Similarly, intermediate ribs 144j nearer to the midpoint of grip portion 145b may have a lesser height than intermediate ribs 144j positioned further from the midpoint of grip portion 145b.

As shown particularly in FIG. 3, some or all of intermediate ribs 144i and intermediate ribs 144j may include an angled surface 144a. Each angled surface 144a may be a radially outwardmost (e.g. outer) surface of respective intermediate ribs 144i, 144j. Angled surfaces 144a may be planar. Angled surfaces 144a may be disposed at a tilt or non-zero angle relative to axis X. In other words, one of a distal edge (e.g., a distalmost end) or a proximal edge (e.g., a proximalmost end) of angled surface 144a may be positioned more radially outward relative to axis X than the other of the distal edge or the proximal edge of angled surface 144a. Explained differently, one of the distal edge or proximal edge of each angled surface 144a may include a greater height than the other of the distal edge or the proximal edge.

For example, as shown in FIG. 3, the intermediate ribs 144i, 144j that are adjacent to one of proximal rib 144p, middle rib 144m, or distal rib 144d may have angled surfaces 144a. Angled surfaces 144a may thus help to define the curved overall profile of grip portions 145a, 145b. Angled surfaces 144a may provide a smoother overall outer profile of grip portions 145a, 145b, decreasing user fatigue and increasing user comfort. More specifically, intermediate rib 144i that is immediately adjacent to distal rib 144d and intermediate rib 144j that is immediately adjacent to middle rib 144m may have an angled surface 144a that is angled proximally (a distal edge of angled surface 144a may be further from axis X than a proximal edge of angled surface 144a). Intermediate rib 144i that is immediately adjacent to middle rib 144m and intermediate rib 144j that is immediately adjacent to proximal rib 144p may have an angled surface 144a that is angled distally (a proximal edge of angled surface 144a may be further from axis X than a distal edge of angled surface 144a).

The following table, Table A, includes exemplary heights and exemplary height ranges for each rib 144 of a first body 130 of medical device 100. As shown, each of the heights and height ranges are in millimeters and are approximate values or ranges. The heights in Table A are measured between axis X and the point shown, perpendicularly to axis X. Thus, a distance from an outer edge of one rib 144 on a first of ribbed sides 142 to an outer edge of a corresponding rib 144 on the second of ribbed sides 142 may be double the distances shown below. Each of distal rib 144d, middle rib 144m, and proximal rib 144d may have a height H1, H2, and H3, respectively. The heights of Table A are indicated in FIG. 3.

TABLE A

| Reference | | | | | | | | | | | |
|---|---|---|---|---|---|---|---|---|---|---|---|
| Number | H1 | H11 | H12 | H13 | H14 | H2 | H21 | H22 | H23 | H24 | H3 |
| First | 8.0-12 | 6.0-10.0 | 5.0-9.0 | 5.0-9.0 | 6.5-10.5 | 9.0-13.0 | 6.5-10.5 | 5.0-9.0 | 6.0-10.0 | 6.5-10.5 | 9.0-13.0 |
| Exemplary | | | | | | | | | | | |
| Height | | | | | | | | | | | |
| Range (mm) | | | | | | | | | | | |
| Second | 9.0-11 | 7.0-9.0 | 6.0-8.0 | 6.0-8.0 | 7.5-9.5 | 10.0-12.0 | 7.5-9.5 | 6.0-8.0 | 6.0-8.0 | 7.5-9.5 | 10.0-12.0 |
| Exemplary | | | | | | | | | | | |
| Height | | | | | | | | | | | |
| Range (mm) | | | | | | | | | | | |
| Exemplary | 10 | 7.9 | 7.0 | 7.1 | 8.6 | 11 | 8.6 | 7.1 | 7.1 | 8.6 | 11 |
| Height (mm) | | | | | | | | | | | |

Still referring to Table A, the concave curve of grip portions 145a and 145b may be similar to one another. For example, heights of the middle two intermediate ribs 144i, 144j may be substantially similar to one another in grip portions 145a and 145b. However, whereas intermediate rib 144i that is adjacent to distal rib 144d may have a greater height than intermediate rib 144i that is adjacent to middle rib 144m, intermediate ribs 144j that are adjacent to middle rib 144m and proximal rib 144m may have the same height. These differences may accommodate different manners in which grip portions 145a and 145b are held. Furthermore, because distal rib 144d has a smaller height than middle rib 144m and proximal rib 144p, the height H11 of intermediate rib 144i that is adjacent to distal rib 144d may provide for a smoother overall curve profile than if the height H11 were the same as the height H14 of intermediate rib 144i that is adjacent to middle rib 144m.

First grip portion 145a may be asymmetrical about a line extending perpendicularly to axis X and bisecting first grip portion 145a. First grip portion 145a may have a steeper curve closer to middle rib 144m than to distal rib 144d. In other words, a proximal portion of first grip portion 145a may be steeper than a distal portion of first grip portion 145a. This asymmetry may be adapted to the manner in which a user grips first grip portion 145a and to accommodate a typical user finger position. Furthermore, the asymmetry may provide a smoother curve profile given the differing heights of proximal rib 144p and middle rib 144m. Second grip portion 145b may be approximately symmetrical about a line extending perpendicularly to axis X and bisecting second grip portion 145b. As shown in the Table A, a difference between the greatest height (e.g., tallest) of first plurality of intermediate ribs 144i and the smallest height (e.g., shortest) of the first plurality of intermediate ribs 144i may be about 1.6 mm. The second plurality of intermediate ribs 144i may include the same difference of about 1.6 mm from the greatest height of the second plurality of intermediate ribs 144j.

Each rib of ribs 144 may include a length (e.g., a dimension extending parallel to axis X). For example, each rib of intermediate ribs 144i and 144j may have a length of approximately 1.5 mm to approximately 3 mm, or a length of approximately 2.0 mm. In aspects, all of intermediate ribs 144i, 144j may have a same length or they may have differing lengths. In an exemplary embodiment, the length of distal rib 144d, middle rib 144m, and proximal rib 144p may be greater than the length of one or more of intermediate ribs 144i, 144j. For example, distal rib 144d, middle rib 144m, and proximal rib 144p may have a length of approximately 1.3 mm to approximately 3.3 m, or a length of approximately 2.3 mm. The length of each of distal rib 144d, middle rib 144m, and proximal rib 144p may be the same length. In some aspects, one or more of distal rib 144d, middle rib 144m, and proximal rib 144p may have a different length than one or more of distal rib 144d, middle rib 144m, and proximal rib 144p.

As shown in FIG. 3, planar sides 160 may be narrower than barrel 132, such that ribs 144 extend alongside portions of barrel 132 on the sides of first body 130 having planar sides 160. In other words, ribs 144 may be wider than barrel 132, such that they extend beyond barrel 132 on sides of first body 130 having planar sides 160.

As shown in FIGS. 1A, 1B, and 3, barrel 132 may extend proximally of a proximalmost of ribs 144. The proximal portion of barrel 132 that extends proximally of ribs 144 may provide a finger rest 146 for at least some users and may facilitate single-handed actuation of medical device 100. In some examples, finger rest 146 may be a boss extending longitudinally along a portion of first body 130. Finger rest 146 may extend distally from a proximal end of first body 130 to middle rib 144m and may intersect with intermediate ribs 144j. First body 130 may include a tapered portion 148 extending distally from distal rib 144d to the distal end of first body 130. Tapered portion 148 may decrease in height as tapered portion 148 extends distally.

Now referring to at least in FIGS. 1B, 2, and 4, second body 180 may include a barrel 182 (e.g. a grip), a shank 184, and an adapter 186. A proximal portion of barrel 182 may serve as a finger rest or gripping surface for the operator's hand. Similar to barrel 132, barrel 182 may have an overall cylindrical shape. In some aspects, a central longitudinal axis of barrel 182 may be approximately coaxial with axis X of handle 120. Shank 184 may be the portion of second body 180 that is movably received within lumen 170 of first body 130.

Adapter 186 may be configured to couple second body 180 to other components of the medical device, such as a fluid source. Adapter 186 may be, for example, a luer adapter and may be at a proximalmost end of second body 180. Second body 180 may further include a lumen 200 (FIG. 2) extending longitudinally through second body 180. Lumen 200 may be configured to receive at least a portion of needle 114 extended therethrough. Adapter 186 may be in fluid communication with the lumen of needle 114 and adapter 186, such that fluid injected through adapter 186 (e.g., a proximal opening of adapter 186) from a fluid source (not shown), such as a syringe, may be delivered to a target site via a distal opening of the lumen of needle 114.

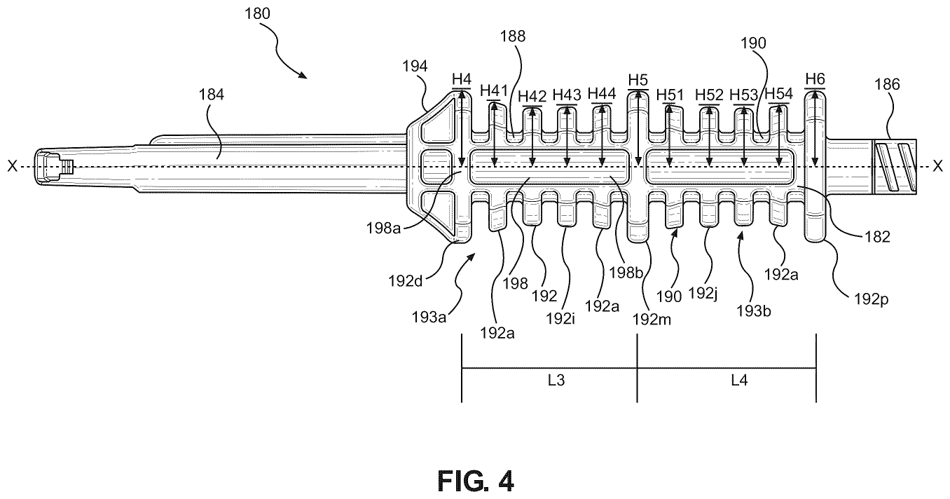

Now referring to FIGS. 1B and 4, barrel 182 may form two recessed sides 198 of second body 180. Recessed sides 198 may include recessed surfaces or depressions and planar surfaces extending along the length of second body 180 parallel to axis X. On two ribbed sides 190 of second body 180, a plurality of ribs 192 may extend radially outward, relative to axis X, from barrel 182. These ribs 192 may provide enhanced grip and tactile feedback for the operator during use of medical device 100, among other benefits. In some aspects, ribbed sides 190 and recessed sides 198 may alternate around the perimeter of second body 180 relative to axis X, creating a varied surface texture that may accommodate different gripping preferences or hand positions. Similar to first body 130, when medical device 100 is positioned on a substantially flat or angled surface, recessed sides 198 and ribbed sides 190 may help to inhibit medical device 100 from rolling on the surface.

Ribbed sides 190 may include ribs 192 extending radially outward from a surface of barrel 182 relative to axis X. Ribs 192 on each of ribbed sides 190 may include a distal rib 192d, a proximal rib 192p, a middle rib 192m, intermediate ribs 192i (e.g., a first plurality of intermediate ribs) positioned between distal rib 192d and middle rib 192m, and intermediate ribs 192j (e.g., a second plurality of intermediate ribs) positioned between middle rib 192m and proximal rib 192p. Similar to first body 130, because each of two ribbed side 190 includes plurality of ribs 192, second body 180 may include two distal ribs 192d, two proximal ribs 192p, two middle ribs 192m, two sets of intermediate ribs 192i (first and third plurality of intermediate ribs), and two sets of intermediate ribs 192j (second and fourth plurality of intermediate ribs). Ribs 192 of first ribbed side 190 may be identical or substantially identical to corresponding ribs 192 of second ribbed side 190. Each rib of ribs 192 may include a planar distal face and a planar proximal face. Together ribs 192 may define a curved outer surface of ribbed sides 190, as described in further detail below. Together, distal rib 192d, middle rib 192m, and intermediate ribs 192i may define a first grip portion 193a, and middle rib 144m, proximal rib 144p, and intermediate ribs 192j may define a second grip portion 193b.

As shown in FIG. 4, distal rib 192d and middle rib 192m may define a distance or length, L3 (approximately parallel to axis X), extending from distal rib 192d to middle rib 192m. Length L3 may be a length of first grip portion 193a. Length L3 (a length of first grip portion 193a) may be about 10 mm to about 30 mm, approximately 15 mm to approximately 25 mm, or approximately 20 mm. Similar to ribs 144, the values of L3 above may optimize a size of grip defined by intermediate ribs 192i, distal rib 192d, and middle rib 192m. For example, these values of L3 may provide good grip to a high number of individuals with vary hand sizes.

Further, middle rib 192m and proximal rib 192p may define a distance or length, L4, extending from middle rib 192m to proximal rib 192p. Length L4 may be a length of a second grip portion 193b. Length L4 (a length of second grip portion 193b) may be about 10 mm to about 30 mm, approximately 15 mm to approximately 25 mm, or approximately 20 mm. The values of L4 above may optimize a size of grip defined by intermediate ribs 192j, middle rib 192m, and proximal rib 192p. For example, these values of L4 may provide good grip to a high number of individuals with vary hand sizes. In examples, lengths L3, L4 (e.g., lengths of grip portions 145a, 145b, respectively) may be the same, to accommodate the similar sizes of the fingers that are typically positioned on grip portion 193a and grip portion 193b.

Similar to first body 130, grip portions 193a and 193b may each have an overall arcuate, concave shape (e.g., a concave outermost surface). Such a shape may result from the varying heights (e.g., an arcuate profile) of intermediate ribs 192i, 192j discussed in further detail below. In other words, first plurality of intermediate ribs 192i may collectively have an arcuate profile that defines a concave outer shape of the grip (e.g., grip portion 193a) and second plurality of intermediate ribs 192j may collectively have an arcuate profile that defines another concave outer shape of the grip (e.g., grip portion 193b). The concave shapes of grip portions 193a and 193b may be chosen to provide a comfortable grip to a wide range of users. For example, users with smaller hands may prefer to hold grip portions 193a or 193b near centers (narrowest) portions of grip portions 193a and 193b, respectively. Additionally or alternatively, users may use the angled proximal or distal portions of grip portions 193a, 193b to provide additional support/a stronger hold on grip portions 193a, 193b. In other words, intermediate ribs 192i nearer to the midpoint of grip portion 193a may have a lesser height than intermediate ribs 192i positioned further from the midpoint of grip portion 193a. Similarly, intermediate ribs 192j nearer to the midpoint of grip portion 193b may have a lesser height than intermediate ribs 192j positioned further from the midpoint of grip portion 193b.

Similar to the configuration of first body 130, some or all of intermediate ribs 192i, 192j may include an angled surface 192a. Each angled surface 192a may be a radially outwardmost (e.g. outer) surface of respective intermediate ribs 192i, 192j. Angled surfaces 192a may be planar. Angled surfaces 192a may be disposed at a tilt or non-zero angle relative to axis X. Explained differently, one of a distal edge (e.g., a distalmost end) or a proximal edge (e.g., a proximalmost end) of angled surface 192a may be positioned more radially outward relative to axis X than the other edge of the distal edge or the proximal edge of angled surface 192a.

As shown in FIG. 4, the intermediate ribs 192i, 192j that are adjacent to one of proximal rib 192p, middle rib 192m, or distal rib 192d may have angled surfaces 192a. Angled surfaces 192a may thus help to define the curved overall profile of grip portions 193a, 193b. Angled surfaces 192a may provide a smoother overall outer profile of grip portions 193a, 193b, decreasing user fatigue and increasing user comfort. More specifically, intermediate rib 192i that is immediately adjacent to distal rib 192d and intermediate rib 192j that is immediately adjacent to middle rib 192m may have an angled surface 192a that is angled proximally (a distal edge of angled surface 192a may be further from axis X than a proximal edge of angled surface 192a). Intermediate rib 192i that is immediately adjacent to middle rib 192m and intermediate rib 192j that is immediately adjacent to proximal rib 192p may have an angled surface 192a that is angled distally (a proximal edge of angled surface 192a may be further from axis X than a distal edge of angled surface 192a).

The following table, Table B, includes exemplary heights and height ranges for each rib 192 of second body 180. Similar to Table A, the heights in Table B are measured between axis X and the point shown, perpendicular to axis X. Thus, a distance from an outer edge of one rib 192 on a first of ribbed sides 190 to an outer edge of a corresponding rib 192 on the second of ribbed sides 190 may be double the distances shown below. The heights and height ranges are provided in millimeters. Each of distal rib 192d, middle rib 192m, and proximal rib 192p may have a height H4, H5, and H6, respectively. As previously discussed, angled surface 192a of some of intermediate ribs 144i, 144j may include first height at the distal edge represented by an odd-numbered reference (e.g., H41, H43) and the second, different height at the proximal edge represented by an even-numbered reference (e.g., H42, H44).

TABLE B

| Reference | | | | | | | | | | | |
|---|---|---|---|---|---|---|---|---|---|---|---|
| Number | H4 | H41 | H42 | H43 | H44 | H5 | H51 | H52 | H53 | H54 | H6 |
| First | 6.0-12 | 5.5-9.5 | 5.0-9.0 | 5.0-9.0 | 5.0-9.0 | 6.0-12 | 5.0-9.0 | 5.0-9.0 | 5.0-9.0 | 5.0-9.0 | 6.0-12 |
| Exemplary | | | | | | | | | | | |
| Height Range | | | | | | | | | | | |
| (mm) | | | | | | | | | | | |
| Second | 8.0-10 | 6.5-8.5 | 6.0-8.0 | 6.0-8.0 | 6.0-8.0 | 8.0-10 | 6.0-8.0 | 6.0-8.0 | 6.0-8.0 | 6.0-8.0 | 8.0-10 |
| Exemplary | | | | | | | | | | | |
| Height Range | | | | | | | | | | | |
| (mm) | | | | | | | | | | | |
| Exemplary | 8.8 | 7.5 | 6.8 | 6.9 | 7.1 | 8.8 | 7.1 | 6.9 | 6.9 | 6.9 | 8.8 |
| Height (mm) | | | | | | | | | | | |

Still referring to Table B, the concave curve of grip portions 193a and 193b may very similar to one another. However, rib 192i that is adjacent to distal rib 192d may have a height that is larger than a height of rib 192j that is adjacent to middle rib 192m, thus creating a slightly different curve profile. This slightly different curve profile may accommodate a manner in which users typically grip first grip portion 193a as opposed to second grip portion 193b.

First grip portion 193a may be asymmetrical about a line extending perpendicularly to axis X and bisecting first grip portion 193a. First grip portion 193a may have a steeper curve closer to distal rib 192d than to middle rib 192m. In other words, a distal portion of first grip portion 193a may be steeper than a proximal portion of first grip portion 193a. Similarly, second grip portion 193b may be asymmetrical about a line extending perpendicularly to axis X and bisecting second grip portion 193b. Second grip portion 193b may have a steeper curve closer to middle rib 192m than to proximal rib 192p. In other words, a distal portion of second grip portion 193b may be steeper than a proximal portion of second grip portion 193b. This asymmetry may be adapted to the manner in which a user grips first grip portion 193a and second grip portion 193b and to accommodate a typical user finger position.

As shown in the Table B, a difference (change) between the greatest height (e.g., tallest) of first plurality of intermediate ribs 192i and the smallest height (e.g., shortest) of the first plurality of intermediate ribs 192i may be approximately 0.2 mm. The second plurality of intermediate ribs 192j may include the same difference of approximately 0.2 mm. Accordingly, the difference of the first or second plurality of intermediate ribs 144i, 144j may be greater compared to the difference of the first or second plurality of intermediate ribs 192i, 192j. In other words, the first or second plurality of intermediate ribs 144i, 144j may have a greater change in height than the first or second plurality of intermediate ribs 192i, 192j. Thus, grip portions 145a and 145b of first body 130 may be more sharply curved than grip portions 193a and 193b of second body 180.

Referring to FIG. 4, recessed sides 198 may include one or more planar surfaces 198a and one or more recessed surfaces 198b (e.g., two recessed surfaces 198b) extending along the length of second body 180 parallel to axis X. Planar surfaces 198a may be narrower than barrel 182, such that ribs 192 extend alongside portion of barrel 182 on side of second body 180 having planar surfaces 198a. Explained differently, and similar to planar sides 160, ribs 192 may be wider than barrel 182 such that they extend beyond barrel 182 on sides of second body 180 having planar surfaces 198a. Each recessed surface 198b of recessed sides 198 may include a concave surface extending along a perimeter of recessed surface 198b. Each planar surface 198a may be positioned between adjacent recessed surfaces 198b. Explained differently, one or more planar surfaces 198a and one or more recessed surfaces 198b may be alternatingly arranged along axis X. Similar to first body 130, when medical device 100 is positioned on a substantially flat or angled surface, planar surfaces 198a of recessed sides 198 may help to prevent medical device 100 from rolling off of the surface.

Each rib of ribs 192 may include a length (e.g., a dimension extending parallel to axis X). For example, each rib of intermediate ribs 192i and 192j may include a length of approximately 1.5 mm to approximately 3 mm, or a length of approximately 2.0 mm. In aspects, all of intermediate ribs 192i, 192j may have a same length or they may have differing lengths. In an exemplary embodiment, the length of distal rib 192d, middle rib 192m, and proximal rib 192p may be greater than the length of one or more of intermediate ribs 192i, 192j. For example, distal rib 192d, middle rib 192m, and proximal rib 192p may have a length of approximately 1.5 mm to approximately 3.5 mm, or approximately 2.5 mm. The length of each of distal rib 192d, middle rib 192m, and proximal rib 192p may be the same length. In some aspects, one or more of distal rib 192d, middle rib 192m, and proximal rib 192p may have a different length than one or more of distal rib 192d, middle rib 192m, and proximal rib 192p.

As shown in FIGS. 1A-1B, and 2, planar sides 160 of first body 130 may include one or more apertures 162. Apertures 162 may be configured to interact with corresponding features on second body 180. For instance, a distal portion of shank 184 of second body 180 may include one or more protrusions 185 (FIG. 2) that are receivable within apertures 162. Protrusions 185 may be biased radially outward relative to axis X or a central longitudinal axis of second body 180 to help facilitate engagement of protrusions 185 within apertures 162. Apertures 162 may include one or more distal apertures 162 and one or more proximal apertures 162, which may allow for multiple engagement positions between first body 130 and second body 180. Apertures 162 and protrusions 185 may function as a locking mechanism to temporarily lock, maintain, or secure medical device 100 in the first or second configuration. In some aspects, the engagement between protrusions 185 and apertures 162 may create a detent mechanism that provides a slight resistance when transitioning between positions, which may help prevent unintended movement of second body 180 relative to first body 130.

As discussed above, medical device 100 may include: a first, distal configuration (as depicted in FIGS. 1A and 2) and a second, proximal configuration (as shown in FIG. 1B). During an exemplary medical procedure using medical device 100, an operator may navigate an insertion device (e.g., an endoscope) such that a distal end of the insertion device is proximate a target site. After navigating the insertion device, the operator may extend sheath 116 and shaft 110 through a working channel of insertion device and an opening of the working channel at the distal end of the insertion device. The operator may extend sheath 116 and shaft 110 through the working channel while medical device 100 is in the second, proximal configuration, so that needle 114 is covered by sheath 116. In the second, proximal configuration, protrusions 185 on shank 184 may engage with proximal apertures 162 of planar sides 160.

The operator may then transition medical device 100 from the second, proximal configuration (e.g., needle 114 is retracted) to the first, distal configuration (e.g., needle 114 is extended) to extend needle 114 distally beyond the distal end of sheath 116. To transition medical device 100, the operator may grip first body 130 and second body 180 with one or more hands and urge second body 180 distally toward first body 130. The arcuate curves of ribs 144, 192 may help to enhance the grip of the operator to prevent slippage. For example, while urging second body 180 toward first body 130, one or more fingers of the operator may contact one or more of the arcuate curves of ribs 144 first body 130 and one or more of the arcuate curves of ribs 144 of second body 180.

After transitioning the medical device to the first, distal configuration, the distal end of needle 114 may be distal to sheath 116. The operator may inject fluid from a fluid source through the lumen of needle 114 via adapter 186 to deliver treatment to tissue at the target site. In the first, distal configuration, protrusions 185 on shank 184 may engage with distal apertures 162 of planar sides 160. After delivering treatment, the operator may transition medical device 100 from the first, distal configuration to the second, proximal configuration to retract needle 114 by gripping first body 130 and second body 180 with one or more hands and urging second body 180 proximally relative to first body 130. The concave curves of first grip portion 145a, 193a and second grip portions 145b, 193b may again help to enhance the grip of the operator to prevent slippage.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Additionally, a variety of elements from each of these embodiments can be combined to achieve a same or similar result as one or more of the disclosed embodiments. Accordingly, the invention is not to be considered as limited by the foregoing description.

We claim:

1. A medical device handle comprising:
a first body defining a lumen; and
a second body having a portion that is movably received within the lumen, wherein the second body includes a grip, wherein the grip includes a proximal rib, a distal rib, a middle rib, a first plurality of intermediate ribs between the proximal rib and the middle rib, and a second plurality of intermediate ribs between the middle rib and the distal rib, wherein each of the first plurality of intermediate ribs and the second plurality of intermediate ribs has an arcuate profile that defines a concave outer shape of the grip,
wherein the second body is configured to move proximally and distally along a central longitudinal axis of the handle in order to actuate a needle of a medical device.

2. The medical device of claim 1, wherein at least some of the first plurality of intermediate ribs have a radially outer surface that is planar and has a non-zero angle with respect to the central longitudinal axis.

3. The medical device of claim 2, wherein each of the first plurality of intermediate ribs and the second plurality of intermediate ribs includes a midpoint, wherein at least one of the angled surfaces of the first plurality of intermediate ribs is angled toward the midpoint of the first plurality of intermediate ribs.

4. The medical device of claim 3, where the radially outer surface a rib of the first plurality of ribs has a proximalmost end that is a first distance from the central longitudinal axis and a distalmost end that is a second distance from the central longitudinal axis, wherein the first distance differs from the second distance.

5. The medical device of claim 1, wherein the second plurality of intermediate ribs collectively have an arcuate profile that defines a concave outer shape of the grip.

6. The medical device of claim 1, wherein the proximal rib, the distal rib, and the middle rib extend further from the central longitudinal axis than any of the intermediate ribs of the first plurality of intermediate ribs and the second plurality of intermediate ribs.

7. The medical device of claim 1, wherein the proximal rib is a first proximal rib, wherein the distal rib is a first distal rib, wherein the middle rib is a first middle rib, wherein the first body includes a second proximal rib, a second distal rib, a second middle rib, a third plurality of intermediate ribs between the second proximal rib and the second middle rib, and a fourth plurality of intermediate ribs between the second middle rib and the second distal rib.

8. The medical device of claim 7, wherein the third plurality of intermediate ribs collectively have an arcuate profile that defines a concave outer shape of the first body.

9. The medical device of claim 7, wherein the fourth plurality of intermediate ribs collectively have an arcuate profile that defines a concave outer shape of the first body.

10. The medical device of claim 7, wherein the third plurality of intermediate ribs of the first body has a greater change in height between tallest and shortest ribs of the third plurality of intermediate ribs compared to a change in height between tallest and shortest ribs of the first plurality of intermediate ribs of the second body.

11. The medical device of claim 7, wherein the second proximal rib and the second distal rib of the first body have different heights, and wherein the first proximal rib and the first distal rib of the second body have a same height.

12. The medical device of claim 7, wherein the second middle rib of the first body has a same height as the second proximal rib of the first body.

13. The medical device of claim 1, wherein a height of a rib of the first plurality of intermediate ribs of the second body that is adjacent to the middle rib is a same height as a rib of the second plurality of intermediate ribs of the second body adjacent to the middle rib.

14. The medical device of claim 1, wherein each of the first plurality of intermediate ribs and the middle rib includes a planar distal face and a planar proximal face.

15. A medical device handle comprising:
a first body defining a lumen, the first body including a plurality of ribs extending radially relative to a central longitudinal axis of the handle, the plurality of ribs including:
a distal rib, a proximal rib, and a middle rib;
a first plurality of intermediate ribs positioned between the distal rib and the middle rib; and
a second plurality of intermediate ribs positioned between the middle rib and the proximal rib, wherein each of the first plurality of intermediate ribs and the second plurality of intermediate ribs has an arcuate profile that defines a concave outer shape of the first body; and
a second body having a portion that is movably received within the lumen;
wherein the second body is configured to move proximally and distally along the central longitudinal axis in order to actuate a needle of a medical device.

16. The medical device handle of claim 15, wherein at least some of the second plurality of intermediate ribs have a radially outer surface that is planar and has a non-zero angle with respect to the central longitudinal axis, wherein the second plurality of intermediate ribs have an arcuate profile that defines a concave curve.

17. The medical device handle of claim 15, wherein each of the distal rib, the proximal rib, and the middle rib extends further from the central longitudinal axis than any of the ribs of the first plurality of intermediate ribs and the second plurality of intermediate ribs.

18. The medical device handle of claim 15, wherein at least some of the first plurality of intermediate ribs and the second plurality of intermediate ribs have a radially outer surface that is planar and has a non-zero angle with respect to the central longitudinal axis.

19. The medical device handle of claim 18, wherein each of the first plurality of intermediate ribs and the second plurality of intermediate ribs includes a midpoint, wherein at least one of the angled surfaces of the first plurality of intermediate ribs is angled toward the midpoint of the first plurality of intermediate ribs.

20. A medical device handle comprising:
a first body defining a lumen, wherein the first body includes: a first plurality of ribs extending radially outward relative to a central longitudinal axis of the handle, wherein the first plurality of ribs forms a first arcuate profile and a second arcuate profile, wherein the second arcuate profile is proximal to the first arcuate profile; and
a second body having a portion that is movably received within the lumen, wherein the second body includes a second plurality of ribs extending radially outward relative to the central longitudinal axis, wherein the second plurality of ribs forms a third arcuate profile and a fourth arcuate profile, wherein the fourth arcuate profile is proximal to the third arcuate profile.
